(19) United States
(12) Patent Application Publication
(10) Pub. No.: US 2026/0306196 A1
(43) Pub. Date: Oct. 8, 2026

(54) PULLWIRE SLACK DETECTION AND ADJUSTMENT FOR FLEXIBLE ELONGATE DEVICES

(71) Applicant: INTUITIVE SURGICAL OPERATIONS, INC., Sunnyvale, CA (US)

(72) Inventors: Shibing Liu, Sunnyvale, CA (US); Samuel B. Schorr, San Jose, CA (US)

(21) Appl. No.: 19/480,985

(22) PCT Filed: May 2, 2024
(86) PCT No.: PCT/US2024/027341

§ 371 Date: Nov. 3, 2025

Related U.S. Application Data
(60) Provisional application No. 63/499,783, filed on May 3, 2023.

(51) Int. Cl. A61B 34/32; A61B 34/00; A61B 34/10; A61B 34/20; A61B 34/30; A61B 34/37; A61B 90/00

(52) U.S. Cl. CPC A61B 34/32; A61B 34/37; A61B 2034/105; A61B 2034/2059; A61B 2034/2061; A61B 2034/301; A61B 2034/715; A61B 2090/062

(57) ABSTRACT

Methods and systems to identify slack in pull wires coupled to an articulable body portion of a flexible elongate device include a control system determining a minimum tension for the pull wires based on a bending parameter of the flexible elongate device.

### FIELD

Disclosed embodiments relate to systems and methods for a flexible elongate device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

### SUMMARY

The following presents a simplified summary of various examples described herein and is not intended to identify key or critical elements or to delineate the scope of the claims.

In accordance with a first example, a medical system is described that includes a flexible elongate device including a pair of pull wires configured to control articulation of the flexible elongate device along an axis. The medical system further includes actuators coupled to the pair of pull wires and configured to apply tensions to the pair of pull wires. A control system of the medical system is configured to determine a bending parameter for the flexible elongate device based on data from one or more sensors, and adjust a minimum tension applied to the pair of pull wires by the actuators based on the bending parameter.

In accordance with a second example, a medical system is described that includes a flexible elongate device having an articulable body portion and a pair of pull wires configured to control articulation of the articulable body portion along an axis. The medical system further includes one or more sensors associated with the flexible elongate device, coupled to the pair of pull wires and configured to apply tensions to the pair of pull wires, and a control system. The control system is configured to determine a measured parameter of the articulable body portion based on data from the one or more sensors, determine an estimated parameter of the articulable body portion based on a state of the one or more actuators, identify slack in at least one of the pair of pull wires based on a comparison of the measured parameter and the estimated parameter, and adjust a minimum tension for the pair of pull wires in response to identifying the slack.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4 is a simplified diagram of a medical system according to some embodiments.

FIG. 5 is a flowchart illustrating a first example method for determining a minimum tension for a medical system according to some embodiments.

FIG. 6 is a flowchart illustrating a second example method for determining a minimum tension for a medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Flexible elongate devices, such as catheters, endoscopes, or other types of flexible instruments, can be maneuvered within a patient to reach a desired treatment location. One type of flexible elongate device includes one or more pairs of opposing pull wires that can be tensioned to control articulation of an articulable body portion of the flexible elongate device, such as a distal section, along an axis (e.g., pitch and/or yaw). Tension in the pull wires is controlled by operation of one or more actuators to increase and decrease tension force on the pull wires. Each pair of pull wires may control articulation along one axis using an antagonistic actuation. Here, the actuators use a minimum tension for a pair of opposing pull wires that corresponds to the lowest tension that is maintained on the pair of pull wires. When the pair of pull wires are at (e.g., the same) minimum tension, the articulable body portion is at a neutral (e.g., straight) position. To perform articulation along the axis controlled by the pair of pull wires, a passive pull wire may be kept at the minimum tension while an active pull wire has its tension changed (e.g., increased or decreased, but maintained at levels higher than or equal to the minimum tension) to cause the articulation. For example, when the articulable body portion is being bent toward a direction, the active pull wire at the side of the articulable body portion corresponding with the bending direction has a tension increase while the passive pull wire on the opposite side of the articulable body portion is kept at the minimum tension. When the articulable body portion is being straightened away from the bending direction, the active pull wire may have a tension decrease while the passive pull wire may be set at the minimum tension (or a higher tension, but not lower than the minimum tension). The use of a minimum tension control scheme helps reduce slack in the pull wire system, increasing responsiveness of actuator control and facilitating effective return of the articulable body portion to a straighter configuration from a more bent configuration.

As a flexible elongate device is inserted into a patient, any bending away from a straight, neutral axis can cause the pull wires to engage a surface (e.g., interior surface of a lumen, axial support structure, etc.) of the flexible elongate device, which causes friction. Accumulated friction that can result from a circuitous path within a patient anatomy and can create slack in the pull wires when the friction force offsets the tension applied to the pull wire by the actuator. While the minimum tension can help reduce slack in the system, accumulated friction can create slack which is undesirable because it can degrade controller performance or can result in incorrect position estimation if using motor encoders for position control. A higher minimum tension is desirable to offset dynamic changes in slack during operation of the flexible elongate device. However, a high minimum tension can stress the pull wires and reduce the useful life of the flexible elongate device. Accordingly, as set forth herein, dynamic adjustment of the minimum tension during a procedure can provide advantages over using a static, predefined minimum tension for the entire procedure.

The methods and systems disclosed herein detect or predict pull wire slack and increase a minimum tension for the pull wire to reduce or eliminate the slack or otherwise counteract the effects of the slack. Slack in the system can be identified or predicted in a number of suitable ways. Generally, slack buildup is a function of the shape of the flexible elongate device, where greater amounts of bending correspond with increased friction and slack. In one example, slack can be determined by comparing the bending angle of the articulable body portion (e.g., as indicated by a shape sensor of the flexible elongate device) with the estimated bending angle of the articulable body portion indicated by encoder data of one or more actuators controlling the pull wire tension. A difference between the bending angle and the estimated bending angle larger than a predetermined threshold can be used to indicate slack in the pull wire. In other or additional examples, pull wire slack can be estimated based on a shape of the flexible elongate device. The shape of the flexible elongate device can correspond to a number and/or severity of bends along the flexible elongate device, a bending angle of the articulable body portion, and/or an insertion length of the flexible elongate device. The shape of the flexible elongate device can be determined by data from one or more sensors, including shape sensors, position sensors, current sensors, torque sensors, and so forth.

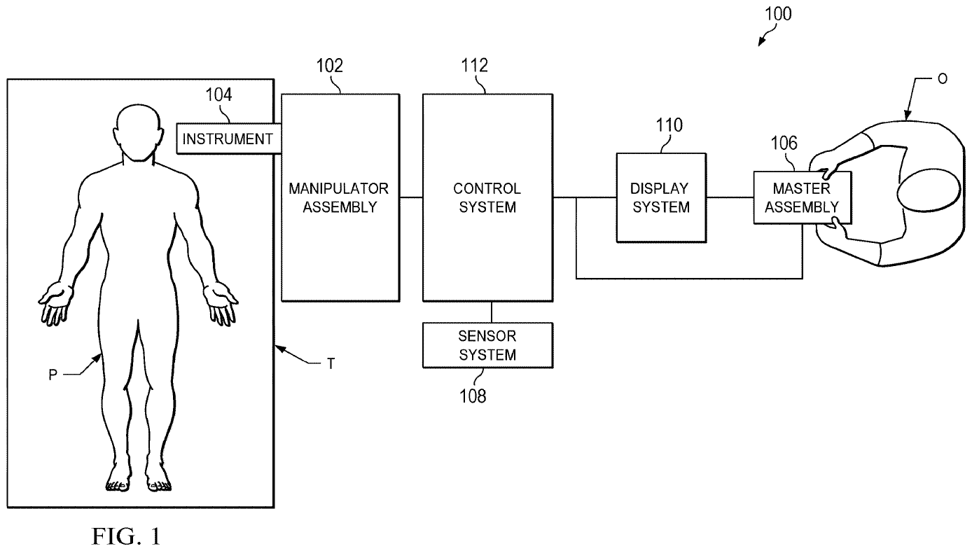

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

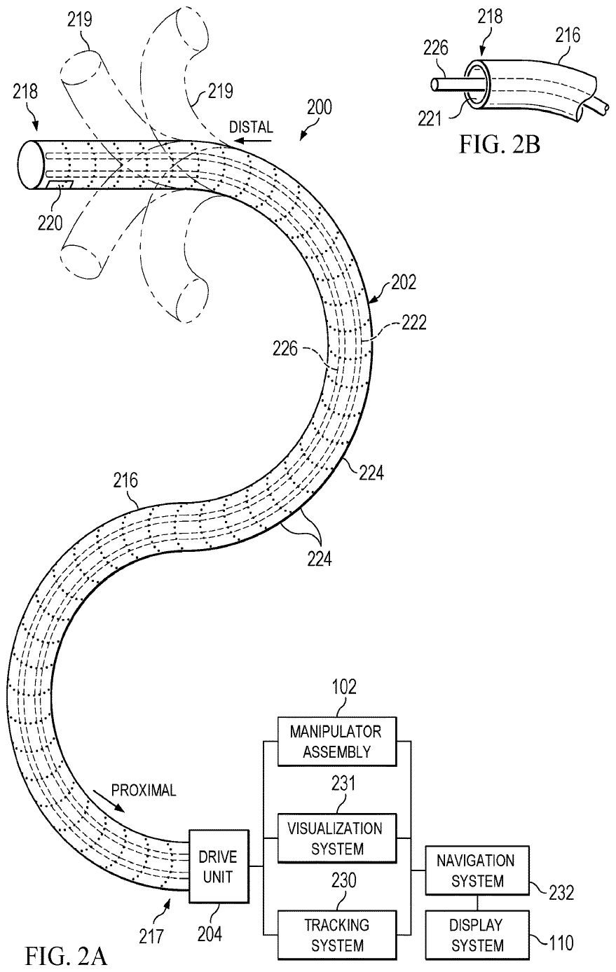

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and arc example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 218. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

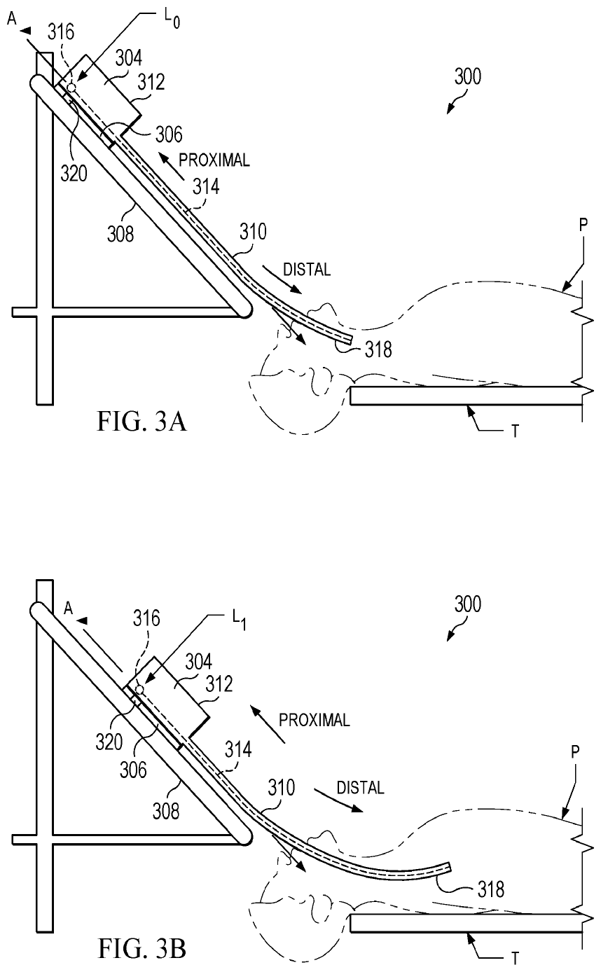

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position LO on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position LO. In some examples, the position L1 may further be used as an indicator of the distance or insertion depth to which the distal end 318 of the elongate device 310 is inserted into the passageway(s) of the anatomy of patient P.

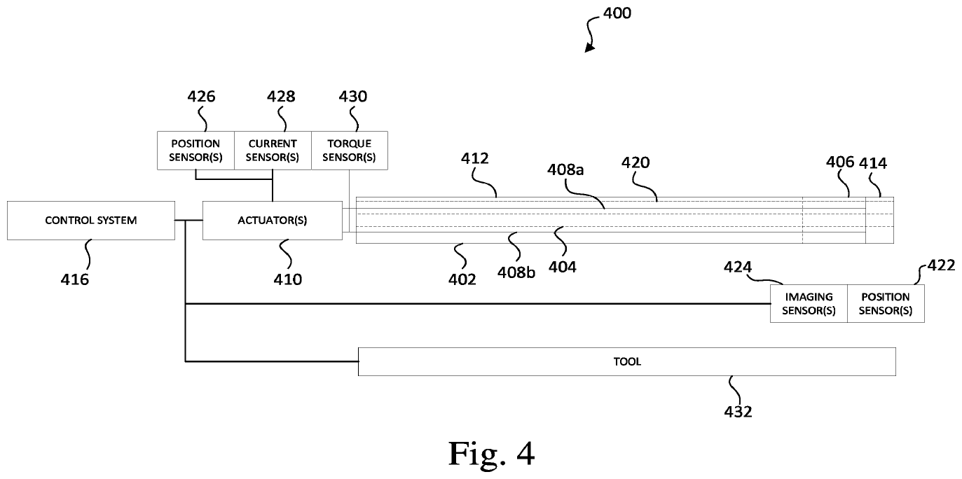

FIG. 4 is a simplified diagram of a medical system 400 that includes a flexible elongate device 402. According to some embodiments consistent with FIGS. 1-3, the medical instrument system 400 may correspond to the medical instrument system 200 and/or the flexible elongate device 402 may correspond to the elongate device 202.

The flexible elongate device 402 has a flexible body that can include a lumen 404 that extends through the flexible body. The lumen 404 may provide a delivery channel for a medical tool, such as a vision probe, a biopsy tool (e.g., a needle, brush, cryoprobe, or forceps), an ablation tool, an electroporation tool, an ultrasound device (e.g., endobronchial ultrasound (EBUS) probe), a chemical delivery tool, and/or the like, to be inserted through the flexible body of the flexible elongate device 402.

As shown, the flexible elongate device 402 includes an articulable body portion 406 and a pair of pull wires 408a, 408b within the articulable body portion 406. One or more actuators 410 are coupled to the pair of pull wires 408a, 408b and are configured to control articulation of the articulable body portion 406 along an axis (e.g., pitch or yaw). Although a pair of pull wires are shown, the flexible elongate device 402 can include one or more additional pairs of pull wires having similar configurations and control as described herein, such that the flexible elongate device 402 may be articulable along multiple axes. In the illustrated example, the body includes a proximal portion 412 and a distal portion 414, where the distal portion 414 includes the articulable body portion 406. The pair of pull wires 408a, 408b can extend along the entire length of the body to couple to the actuator(s) 410 adjacent to the proximal portion 412 of the flexible elongate device 402.

The medical system 400 further includes a control system 416 operably coupled to the actuator(s) 410 to control operation thereof to thereby control articulation of the articulable body portion 406. So configured, a user may utilize the control system 416 to maneuver the flexible elongate device 402 through the anatomy of a patient, resulting in the body of the flexible elongate device 402 having bends along the length thereof.

As shown in FIG. 4, the flexible elongate device 402 may further include one or more sensors coupled thereto or associated therewith to provide data regarding the control, status, position, orientation, speed, velocity, pose, and/or shape of the flexible elongate device 402. For example, the flexible elongate device 402 may include a shape sensor 420 (e.g., fiber optic shape sensor) extending along a length thereof that provides data regarding the shape and position of the flexible elongate device 402, including the articulable body portion 406 (e.g., position and/or bending angle) thereof. In additional or alternative examples, the flexible elongate device 402 may include one or more position sensors 422 (e.g., electro-magnetic sensors) and/or one or more imaging sensors 424 (e.g., camera, ultrasound, fluoroscope, etc.). Although shown in FIG. 4 as being separate from the flexible elongate device 402, the position sensors 422 and/or imaging sensors 424 may be integrated with the flexible elongate device 402, or a tool that is inserted through the lumen 404 of the flexible elongate device 402. In some embodiments, a shape sensor 420 may be additionally or alternatively located in a tool that is inserted through the lumen 404 of the flexible elongate device 402.

The system 400 can further include one or more sensors associated with the actuator(s) 410 to provide data regarding the operation thereof. For example, the system 400 can include one or more actuator position sensors 426, such as resolvers, encoders, potentiometers, and the like, that describe the rotation and/or orientation of the actuator(s) 410.

The system 400 can further include one or more sensors configured to provide data regarding a tension of the pull wires 408a, 408b. In one example, the system 400 includes one or more current sensors 428 configured to measure a current through a motor of the actuator(s) 410. In this example, the system 400 can be implemented without using torque sensors to directly measure pull wire tension. In another example, the system 400 includes one or more torque sensors 430 coupled to an output of a gearbox of the actuator(s) 410 and/or coupled to one or both of the pull wires 408a, 408b. The sensor(s) 428, 430 allow the control system 416 to track tension in the pull wires 408a, 408b.

According to some embodiments, the control system 416 can be configured to determine a bending parameter for the flexible elongate device 402 based on sensor data. The bending parameter is a metric or value that is dependent on the curvature of the flexible elongate device 402, and thus correlated with the amount of slack in the pull wires of the flexible elongate device 402. The bending parameter may provide an indication of the severity and/or cumulative nature of bends that the flexible elongate device 402 has due to maneuvering within a patient's anatomy, which provides information regarding potential friction within flexible elongate device 402 and the resulting slack created in the pull wires 408a, 408b extending therethrough.

The bending parameter can include any desired number of inputs. In some examples, the bending parameter can include an overall or partial shape of the flexible elongate device 402 provided by data from the shape sensor 420; an accumulated curvature along a portion (e.g., some or all of the articulable body portion 406, a distal portion, an intermediate portion, and/or a proximal portion) or an entire length of the flexible elongate device 402 based on data from the shape sensor 420, the position sensor(s) 422, and/or the imaging sensors 424; accumulated bending angles of the articulable body portion 406 of the flexible elongate device 402 based on data from the shape sensor 420, the position sensor(s) 422, and/or the imaging sensor(s) 424; an insertion depth of the flexible elongate device 402 into a patient's anatomy based on data from the shape sensor 420, the position sensor(s) 422, the imaging sensors 424, and/or system-side sensors, such as optical sensors or sensors coupled to drives or structure moving the flexible elongate device 402 forward; and/or an anatomical model of the patient anatomy, which can include an intended path within the patient anatomy for a particular procedure.

Each of the above inputs is directed at determining the number and/or severity of bends (e.g., deviation of a portion of the flexible elongate device from a linear orientation) in the flexible elongate device 402 at a given point during a procedure. For example, a larger number of bends and/or smaller bend radius (e.g., for any bend) may correlate with a larger bending parameter while a smaller number of bends and/or larger bend radius may correlate with a smaller bending parameter. Here, a larger bending parameter correlates with a greater amount of slack. In one example, the bending parameter can be based on a count of the number of bends in the flexible elongate device 402, either dynamically based on data from the shape sensor 420 or based on an accumulated count of times that the articulable body portion 406 is maneuvered during a procedure based on data from the shape sensor 420, position sensor(s) 422, and/or the imaging sensor(s) 424. In an additional or alternative example, the bending parameter can be based on an estimated count of the number of bends in the flexible elongate device 402. The estimate can be based on an insertion depth of the flexible elongate device 402 within a patient, where it is assumed that the further the flexible elongate device 402 is inserted into a patient, the more bends and/or higher severity of bends will be accumulated, and/or an anatomical model of the patient anatomy, which can determine the total number of bends and/or severity of bends that will be imparted on the flexible elongate device 402 during a procedure based on an intended path through the patient anatomy.

In an additional or alternative example, the bending parameter can be a difference between an expected parameter (e.g., position, bending radius, and/or bending angle) of the articulable body portion 406 of the flexible elongate device 402 (e.g., as determined by predictive modeling) and an actual parameter (e.g., position, bending radius, and/or bending angle) of the articulable body portion 406 of the flexible elongate device 402 (e.g., as measured by one or more sensors). The amount of difference between the predicted and actual parameters may be caused by the amount of slack.

In one embodiment, the system 400 includes the actuator position sensor 426. Based on data from the actuator position sensor 426 (e.g., encoder data), an expected parameter of the articulable body portion 406 of the flexible elongate device 402 can be determined based on modeling of the system 400, where operation of the actuator 410 is correlated to movement of the articulable body portion 406. In instances where there is slack in one or both of the pull wires 408a, 408b, the actual parameter of the articulable body portion 406 will be different from the expected parameter because the full input of the actuator 410 is not imparted on the articulable body portion 406 due to the slack. To obtain the actual parameter of the articulable body portion 406, the system 400 can use the shape sensor 420 and/or the position sensor 422.

For system utilizing estimated and actual position to at least partially determine the bending parameter, the difference threshold can be between about 3 mm and about 5 mm, or between about 3 mm and about 10 mm.

For systems utilizing estimated and actual bending angle to at least partially determine the bending parameter, the difference threshold can be between about 30 degrees and about 90 degrees, between about 40 degrees and about 80 degrees, between about 50 degrees and about 70 degrees, or about 60 degrees.

For systems utilizing estimated and actual bending radius to at least partially determine the bending parameter, the difference threshold can be between about 5 mm and about 8 mm, or between about 5 mm and about 15 mm.

The control system 416 operates the actuator(s) 410 to maintain a minimum tension in the pair of pull wires 408a, 408b. For an antagonistic pair of pull wires 408a, 408b that control a single axis of articulation, minimum tension refers to a lowest allowable tension for either pull wire 408a or pull wire 408b. For example, the minimum tension may correspond to a tension that is applied to a passive pull wire of the pair of pull wires 408a, 408b during an articulation change of the articulable body portion 406 caused by a tension change applied to an active pull wire of the pair of pull wires 408a, 408b. Here, the active pull wire has increased tension above the minimum tension to control articulation while the passive pull wire is maintained at the minimum tension. The minimum tension may be dynamically adjusted to ensure that tensions applied to the pull wires 408a, 408b do not fall below a bottom threshold required or intended for the system 400. Tension changes applied to the active pull wire 408a, 408b for an articulation movement can include an increase or decrease in tension that is maintained at higher or equal to the minimum tension.

In some examples, the system 400 may use a lowest possible minimum tension and/or a highest possible minimum tension as bounds for the dynamically adjustable minimum tension. These values may be included to ensure proper operation of the system 400 and/or a desired lifespan for the components of the system 400. In these examples, the control system 416 would stop decreasing the minimum tension when reaching the lowest possible minimum tension and would stop increasing the minimum tension when reaching the highest possible minimum tension.

In some embodiments, the control system 416 determines the bending parameter for the flexible elongate device 402 during a procedure and, based on the bending parameter, adjusts the minimum tension in the pull wires 408a, 408b accordingly. The adjustment of the minimum tension can include increasing the minimum tension and/or lowering the minimum tension. In some examples, the control system 416 can be configured to lower the minimum tension (e.g., a predetermined amount or to the lowest possible minimum tension) after reaching a desired location within a patient for a particular procedure, such that no further maneuvering of the articulable body portion 406 is intended. The control system 416 can determine arrival at the desired location by any suitable method, including, for example, a mode change in the system 400, seating of a drive for the flexible elongate device 402, based on data from a position/shape sensor, and so forth.

In order to try to overcome friction in the flexible elongate device 402 and remove slack in the pull wires 408a, 408b, the control system 416 can determine a new minimum tension (e.g., an increased minimum tension from a previous minimum tension) for the pair of pull wires 408a, 408b based on the bending parameter. The determination can also include an intermediate step that determines slack in one or both of the pull wires 408a, 408b based on the bending parameter and subsequently determining the minimum tension needed to remove the slack. Thereafter, the control system 416 controls the operation of the actuator(s) 410 in accordance with the determination using the minimum tension for the pull wires 408a, 408b.

The determination of the new minimum tension can follow any number of suitable decision paths. For example, the determination can correspond to a preset increase of the minimum tension in response to the bending parameter being greater than a predetermined threshold. In another example, the bending parameter can have a plurality or series of increasing threshold values (e.g., stepped change) with a corresponding series of increasing values for the minimum tension. In yet another example, the bending parameter can have values disposed along a scale (e.g., linear or some other relationship) with correspondingly scaled increases to the minimum tension.

In some examples, the control system 416 implements the new minimum tension and awaits further data to determine whether an additional increased in the minimum tension is needed. In other examples, the control system 416 increases the minimum tension until the control system determines that the slack is removed from the pull wires 408a, 408b.

A determination that the slack is removed from the pull wires 408a, 408b, can be done in a number of suitable ways. In one example, the control system 416 performs the comparison between the expected parameter of the articulable body portion 406 and the parameter of the articulable body portion 406 to determine the bending parameter, as discussed in more detail above. With this comparison, the control system 416 can monitor the difference between the expected parameter and the actual parameter of the articulable body portion 406 as the minimum tension is increased and stop increasing the minimum tension in response to the difference falling below a predetermined threshold.

In other examples, the control system 416 may monitor the motion of the flexible elongate device 402 and use motion smoothness as an indicator of when the slack is removed from the pull wires 408a, 408b. Removal of slack (e.g., not smooth motion) can be identified by oscillation of the articulable body portion 406, one or more sudden jumps shown in position data for the articulable body portion 406, and so forth. In these examples, on identification of data indicating removal of slack, the control system 416 stops increasing the minimum tension.

In some embodiments, the control system 416 determines the bending parameter for the flexible elongate device 402 during a procedure and, based on the bending parameter, detects slack in the pull wires 408a, 408b. The control system 416 then determines the minimum tension in response to the slack. The determination of the minimum tension can include increasing the minimum tension. In some examples, the control system 416 can determine the minimum tension based on the bending parameter utilized to detect slack in the pull wires 408a, 408b or based on a second bending parameter different from the bending parameter utilized to detect slack in the pull wires 408a, 408b. For example, the first bending parameter can be based on one or more of the inputs described above and the second bending parameter can be based on one or more different inputs or different combinations of the inputs.

As discussed above, the flexible elongate device 402 includes the lumen 404 that provides a delivery channel for a medical tool 432, such as a vision probe, a biopsy tool (e.g., a needle, brush, cryoprobe, or forceps), an ablation tool, an electroporation tool, an ultrasound device (e.g., endobronchial ultrasound (EBUS) probe), a chemical delivery tool, and/or the like. The properties of the tool 432 can impact the properties and shape of the flexible elongate device 402 and, as a result, can change the friction within the flexible elongate device 402. Accordingly, in further embodiments, the control system 416 is configured to adjust the bending parameter based on the tool 432 inserted or to be inserted into the lumen 404 for a particular procedure. For example, the presence of a tool may result in an increase in friction and slack, and thus a higher minimum tension. Identification of the tool 432 can be input by a user of the system 400 and/or can be identified based on sensor identification (e.g., induction sensors providing inductance data along a length of the tool 432 to be matched to inductance data of a known tool type). In some examples, the properties of the tool or the impact caused by the properties (e.g., stiffness, friction, etc.) may be used to adjust the minimum tension.

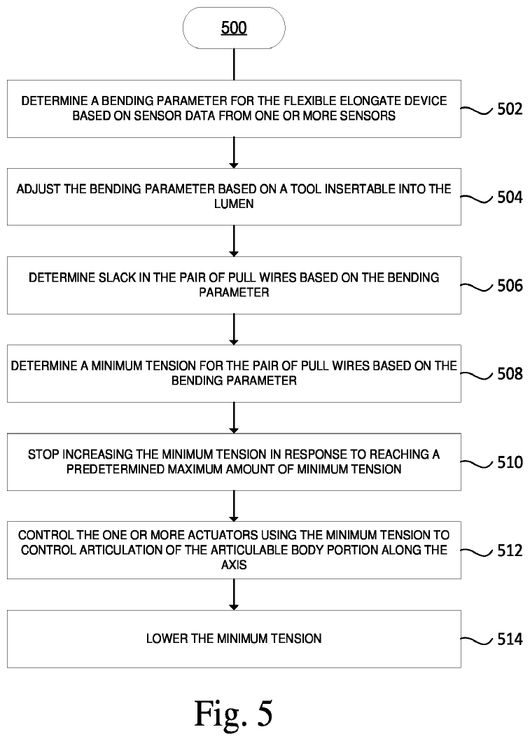

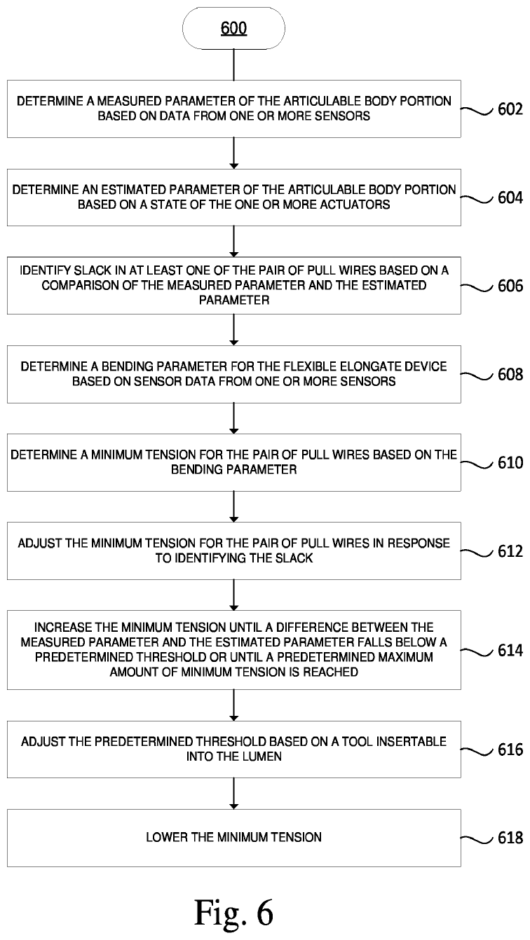

FIG. 5 illustrates a method 500 for determining a minimum tension for a medical system including a flexible elongate device (e.g., the medical system 400 and flexible elongate device 402) according to some embodiments. The method 500 is illustrated as a set of operations or processes 502 through 514. Not all of the illustrated processes may be performed in all embodiments of method 500. Additionally, one or more processes that are not expressly illustrated in FIG. 6 may be included before, after, in between, or as part of the processes 502 through 514. Processes may also be performed in different orders. In some embodiments, one or more of the processes 502 through 514 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 502 through 514 may be performed by a controller (e.g., control system 416).

In process 502, a bending parameter for a pair of pull wires (e.g., pull wires 408a, 408b) of a flexible elongate device (e.g., flexible elongate device 402) is determined by a control system (e.g., control system 416) based on data from one or more sensors (e.g., sensors 420, 422, 424, 426). In process 504, the bending parameter may be adjusted based on a tool (e.g., tool 432) insertable into a lumen (e.g., lumen 404) of the flexible elongate device. In process 506, slack in the pair of pull wires is determined based on the bending parameter.

In process 508, a minimum tension for the pair of pull wires is determined based on the bending parameter. In some examples, determining the minimum tension in process 508 can include determining a minimum tension sufficient to remove the slack, providing an increasing minimum tension to remove the slack in response to determining that the bending parameter is greater than a predetermined threshold, and/or providing an increasing minimum tension to remove the slack based on motion smoothness of the flexible elongate device. In process 510, the control system may stop increasing the minimum tension in response to reaching a predetermined maximum amount of minimum tension. In process 512, the control system controls one or more actuators (e.g., actuators 410) coupled to the pair of pull wires using the minimum tension to control articulation of an articulable body portion (e.g., articulable body portion 406) of the flexible elongate device along an axis. In process 514, the control system lowers the minimum tension.

In some examples, process 514 can include: lowering the minimum tension after reaching a desired location within a patient (e.g., at a target or other location where further articulation of the articulable body portion is not expected); lowering the minimum tension in response to a decrease in the bending parameter (e.g., due to partial retraction, relocation, patient movement, or other movement of the flexible elongate device; due to insertion of a tool increasing the combined stiffness of the tool and flexible elongate device, etc.); and/or lowering the minimum tension when the flexible elongate device is retracted at the end of a procedure.

FIG. 6 illustrates a method 600 for determining a minimum tension for a medical system including a flexible elongate device (e.g., the medical system 400 and flexible elongate device 402) according to some embodiments. The method 600 is illustrated as a set of operations or processes 602 through 618. Not all of the illustrated processes may be performed in all embodiments of method 600. Additionally, one or more processes that are not expressly illustrated in FIG. 6 may be included before, after, in between, or as part of the processes 602 through 618. Processes may also be performed in different orders. In some embodiments, one or more of the processes 602 through 618 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 602 through 618 may be performed by a controller (e.g., control system 416).

In process 602, a measured parameter of an articulable body portion (e.g., articulable body portion 406) of a flexible elongate device (e.g., flexible elongate device 402) is determined by a control system (e.g., control system 416) based on data from one or more sensors (e.g., sensors 420, 422, 424, 426). In process 604, an estimated parameter of the articulable body portion of the flexible elongate device is determined by the control system based on a state of one or more actuators (e.g., actuators 410) coupled to a pair of pull wires (e.g., pull wires 408a, 408b) of the flexible elongate device. In some examples, the measured parameter can be a measured position, bending angle, and/or bending radius of the articulable body portion and the estimated parameter can be an estimated position, bending angle, and/or bending radius of the articulable body portion.

In process 606, the control system identifies slack in at least one of the pair of pull wires based on a comparison of the measured parameter and the estimated parameter. In process 608, the control system determines a bending parameter for the flexible elongate device based on data from one or more sensors (e.g., sensors 420, 422, 424, 426). In process 610, the control system determines a minimum tension for the pair of pull wires based on the bending parameter. In process 612, the control system adjusts the minimum tension for the pair of pull wires in response to identifying the slack. In process 614, the control system increases the minimum tension until a difference between the measured parameter and the estimated parameter falls below a predetermined threshold or until a predetermined maximum amount of minimum tension is reached. In process 616, the control system adjusts the predetermined threshold based on a tool (e.g., tool 432) insertable into a lumen (e.g., lumen 404) of the flexible elongate device. In process 618, the control system lowers the minimum tension.

In some examples, process 618 can include lowering the minimum tension after reaching a desired location within a patient (e.g., at a target or other location where further articulation of the articulable body portion is not expected); and/or lowering the minimum tension based on the estimated and measured parameters (e.g., a difference between the estimated and measured parameters is zero or lower than a predetermined threshold, or in response to the estimated and measured parameters being consistent).

One or more components of the embodiments discussed in this disclosure, such as control system 112, 416, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system, comprising:
a flexible elongate device, including a pair of pull wires configured to control articulation of the flexible elongate device along an axis;
actuators coupled to the pair of pull wires and configured to apply tensions to the pair of pull wires; and
a control system configured to:
determine a bending parameter for the flexible elongate device based on data from one or more sensors; and
adjust a minimum tension applied to the pair of pull wires by the actuators based on the bending parameter.

2. The medical system of claim 1, wherein the bending parameter comprises a shape of at least a portion of the flexible elongate device.

3. The medical system of claim 1, wherein the bending parameter comprises an accumulated curvature along at least a portion of the flexible elongate device.

4. The medical system of claim 1, wherein the bending parameter comprises at least one of:
accumulated bending angles along at least a portion of the flexible elongate device; or
a radius of curvature along at least a portion of the flexible elongate device.

5. The medical system of claim 1, wherein the bending parameter is based on one or more of: insertion depth of the flexible elongate device; or an anatomical model of anatomy of a patient.

6. The medical system of claim 1, wherein:
the flexible elongate device includes an articulable body portion and the pair of pull wires are configured to control articulation of the articulable body portion; and
the bending parameter comprises a difference between an expected parameter and a measured parameter of the articulable body portion.

7. The medical system of claim 6, wherein the expected parameter is at least one of:
an expected bending angle and the measured parameter is a measured bending angle;
an expected position and the measured parameter is a measured position; or
an expected bending radius and the measured parameter is a measured bending radius.

8.-9. (canceled)

10. The medical system of claim 6, further comprising one or more actuator encoders coupled to the actuators; and wherein the flexible elongate device includes a shape sensor, the expected parameter is determined using the one or more actuator encoders, and the measured parameter is determined using the shape sensor.

11. The medical system of claim 6, wherein the control system is further configured to:
identify slack in the pair of pull wires based on the bending parameter; and
remove the slack in the pair of pull wires by increasing the minimum tension until the difference between the expected parameter and the measured parameter of the articulable body portion falls below a predetermined threshold.

12. The medical system of claim 1, wherein the control system configured to adjust the minimum tension applied to the pair of pull wires by the actuators based on the bending parameter includes the control system being configured to:
identify slack in the pair of pull wires based on the bending parameter; and
determine the minimum tension in response to the slack.

13. The medical system of claim 12, wherein the control system is configured to determine the minimum tension based on a second bending parameter different from the bending parameter.

14. The medical system of claim 12, wherein the control system is configured to remove slack by:
increasing the minimum tension until the slack is removed based on motion smoothness of the flexible elongate device;
increasing the minimum tension in a series of step changes; or
increasing the minimum tension in a linear transition.

15. The medical system of claim 1, wherein the control system is configured to lower or increase the minimum tension based on the bending parameter.

16. The medical system of claim 1, wherein the system does not include any torque sensors configured to directly measure tension in the pair of pull wires.

17. The medical system of claim 1, wherein the flexible elongate device comprises a lumen extending therethrough for receiving one or more tools; and the control system is configured to determine the minimum tension based on a tool insertable into the lumen.

18.-34. (canceled)

35. A non-transitory computer readable medium having instructions stored thereon that, when executed by a computing device, cause the computing device to:
determine a bending parameter for a flexible elongate device based on data from one or more sensors; and
adjust a minimum tension applied to a pair of pull wires configured to control articulation of the flexible elongate device by actuators coupled to the pair of pull wires based on the bending parameter.

36. The non-transitory computer readable medium of claim 35, wherein the bending parameter comprises or is based on one or more of: a shape of at least a portion of the flexible elongate device; an accumulated curvature along at least a portion of the flexible elongate device; accumulated bending angles along at least a portion of the flexible elongate device; a radius of curvature along at least a portion of the flexible elongate device; insertion depth of the flexible elongate device; or an anatomical model of anatomy of a patient.

37. The non-transitory computer readable medium of claim 35, wherein to adjust the minimum tension applied to the pair of pull wires by the actuators based on the bending parameter, the instructions, when executed, cause the computing device to:
identify slack in the pair of pull wires based on the bending parameter; and
determine the minimum tension in response to the slack.

38. The non-transitory computer readable medium of claim 37, wherein the instructions, when executed, further cause the computing device to:
determine the minimum tension based on a second bending parameter different from the bending parameter; or
determine the minimum tension based on a tool insertable into a lumen extending through the flexible elongate device.

39. The non-transitory computer readable medium of claim 35, wherein the instructions, when executed, further cause the computing device to:
lower or increase the minimum tension based on the bending parameter.

40.-45. (canceled)
